(19) United States
(12) Patent Application Publication
(10) Pub. No.: US 2022/0175586 A1
(43) Pub. Date: Jun. 9, 2022

(54) Heat-shrinkable medical dressing structure for suture-free and scar-free wound closure

(71) Applicant: Zhijun PENG, Foshan (CN)

(72) Inventor: Zhijun PENG, Foshan (CN)

(21) Appl. No.: 17/540,223

(22) Filed: Dec. 1, 2021

(30) Foreign Application Priority Data
Dec. 5, 2020 (CN) 202011404428.4

(51) Int. Cl. A61F 13/02; A61F 15/00

(52) U.S. Cl. CPC A61F 13/0236; A61F 2013/00468; A61F 13/025; A61F 15/007

(57) ABSTRACT

A heat-shrinkable medical dressing structure for suture-free and scar-free wound closure, including a heat-shrinkable sheet portion and a non-heat-shrinkable sheet edge portion. The non-heat-shrinkable sheet edge portion is arranged along the bottom peripheral surface of the heat-shrinkable sheet portion and the two portions are fixed as one. The bottom surface of the non-heat-shrinkable sheet edge portion is provided with an adhesive layer. Shrinking force, produced when the heat-shrinkable sheet portion is heated, interworks with the adhesive edges so that wound closure is easily achieved without any sutures or secondary wounds. The heat-shrinkable medical dressing structure helps lessen pain and there will not be any scarring from sutures. It is easy to use and saves time and effort.

### BACKGROUND OF THE INVENTION

The present invention relates to wound closure products and more particularly pertains to a medical dressing for wound closure.

Adhesive bandage is an essential first aid medical item most commonly used in hospitals, clinics and households. It is an elongated plaster in which a piece of gauze immersed in drug solution is attached to the center of the plaster. An adhesive bandage is adhered to the wound to protect it from bleeding, to resist the growth of bacteria and to prevent further injuries. Yet, the adhesive bandage can only hide the wound, prevent the wound from further injuries and protect the wound from the attack by bacteria from the external environment; it does not sew up the wound for faster healing nor leaves a smaller scar after the wound heals. In the present art, medical suture needles and threads are adopted for sewing up wounds. The operating process takes time, creates a secondary wound and requires removal of sutures after the wound heals, which all add metal stress and physical pain to the patient. There is no other wound closure products which are convenient to use and pain-relieving.

### BRIEF SUMMARY OF THE INVENTION

In view of the aforesaid disadvantages now present in the prior art, the present invention provides a heat-shrinkable medical dressing structure for suture-free and scar-free wound closure. The shrinking force, produced when the heat-shrinkable sheet portion is heated, interworks with the adhesive edges so that wound closure is easily achieved without any sutures or secondary wounds. The present invention helps lessen pain and there will not be any scarring from sutures after the wound heals. It is easy and convenient to use and saves time and effort.

To attain this, the technical solution of the present invention adopts the following three schemes:

The first scheme: a heat-shrinkable medical dressing structure for suture-free and scar-free wound closure, which comprises a heat-shrinkable sheet portion and a non-heat-shrinkable sheet edge portion. The non-heat-shrinkable sheet edge portion is arranged along the bottom peripheral surface of the heat-shrinkable sheet portion and the two portions are fixed as an integral structure. The bottom surface of the non-heat-shrinkable sheet edge portion is provided with an adhesive layer.

The second scheme: a heat-shrinkable medical dressing structure for suture-free and scar-free wound closure, which comprises a plurality of heat-shrinkable sheet portions and a pair of non-heat-shrinkable sheet edge portions. The plurality of heat-shrinkable sheet portions are arranged laterally between the pair of non-heat-shrinkable sheet edge portions, and are equidistantly spaced apart from one another. The two end portions of each of the heat-shrinkable sheet portions are fixed respectively with the pair of non-heat-shrinkable sheet edge portions; there is a separating hole between every two adjacent heat-shrinkable sheet portions; the bottom surface of each of the non-heat-shrinkable sheet edge portions is provided with an adhesive layer. Therefore, a roll of medical dressing is formed.

The third scheme: a heat-shrinkable medical dressing structure for suture-free and scar-free wound closure, which comprises a heat-shrinkable sheet portion and a pair of non-heat-shrinkable sheet edge portions. The heat-shrinkable sheet portion is in rectangular shape; the pair of non-heat-shrinkable sheet edge portions are arranged on the two end portions of the heat-shrinkable sheet portion respectively and fixed with the heat-shrinkable sheet portion as a one whole piece. One side of each of the non-heat-shrinkable sheet edge portions is provided with an adhesive layer.

Based on the first scheme, the preferred embodiment are as follows:

Preferably, the bottom peripheral surface of the heat-shrinkable sheet portion corresponding to inner edges the non-heat-shrinkable sheet edge portion is provided with shrinkage holes.

Preferably, the bottom surface of the heat-shrinkable sheet portion is provided with an isled compound padding.

Preferably, the inner edges of the non-heat-shrinkable sheet edge portion fixed with the heat-shrinkable sheet portion are provided with shrinkage notches.

The benefits of the present invention: The shrinking force, produced when the heat-shrinkable sheet portion is heated, interworks with the adhesive edges so that wound closure is easily achieved without any sutures or secondary wounds. The present invention helps lessen pain and does not leave any scars formed from sutures after the wound is healed. It is easy and convenient to use and saves time and effort.

### BRIEF DESCRIPTION OF THE DRAWINGS

FIG. 1 shows the schematic perspective view of a first implementing example of the present invention.

FIG. 2 shows the cross-sectional view of the first implementing example of the present invention, modified with an additional pair of detachable sheets.

FIG. 3 shows the schematic perspective view of a second implementing example of the present invention.

FIG. 4 shows the cross-sectional view of the second implementing example of the present invention, modified with an additional pair of detachable sheets.

FIG. 5 shows the schematic front view of a third implementing example of the present invention.

FIG. 6 shows the cross-sectional view of the third implementing example of the present invention, modified with an additional pair of detachable sheets.

FIG. 7 shows the schematic perspective view of a third implementing example of the present invention in a rolled manner.

FIG. 8 shows the partial perspective view of a fourth implementing example of the present invention.

FIG. 9 shows the schematic perspective view of a fifth implementing example of the present invention.

### DETAILED DESCRIPTION OF THE INVENTION

### The First Embodiment

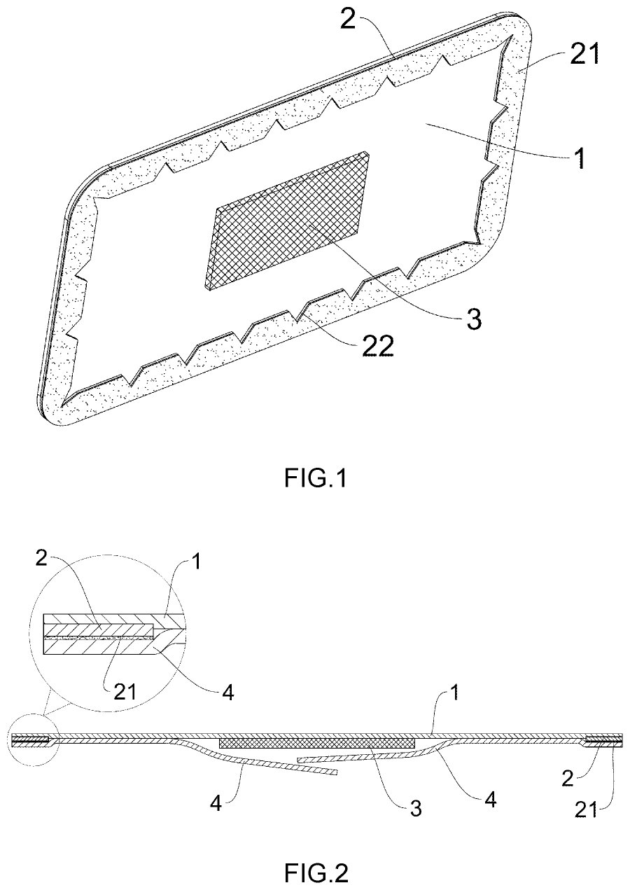

As illustrated in FIG. 1 to FIG. 4, a heat-shrinkable medical dressing structure for suture-free and scar-free wound closure, comprises a heat-shrinkable sheet portion 1 and a non-heat-shrinkable sheet edge portion 2. The non-heat-shrinkable sheet edge portion 2 is arranged along the bottom peripheral surface of the heat-shrinkable sheet portion 1 and the two portions are fixed as an integral structure. The bottom surface of the non-heat-shrinkable sheet edge portion 2 is provided with an adhesive layer 21. When using, adhere the present invention on the wound through the adhesive layer 21 and then increase the temperature of the heat-shrinkable sheet portion 1 to an appropriate level (for example, blow hot air to the heat-shrinkable sheet portion using a hair dryer), so that the heat-shrinkable sheet portion 1 physically shrinks, pulls the muscles around the wound to contract in a centripetal manner and achieves wound closure, which is similar to the effect of using sutures. When the wound heals, tear off the dressing and there will not be any scarring from sutures. The non-heat-shrinkable sheet edge portion 2 is manufactured from soft plastic with a certain level of heat resistance and the heat-shrinkable sheet portion 1 is manufactured from heat-shrinkable plastic. The heat-shrinkable sheet portion 1 is 7 μm-10 μm thick, manufactured from thin film material with a shrinking performance of 70%.

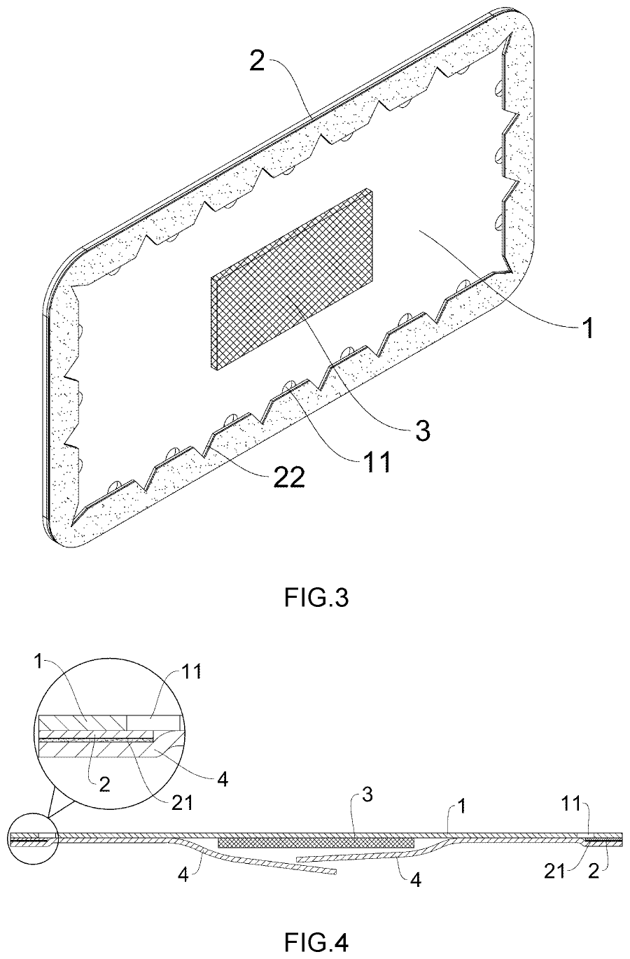

To achieve more regular shrinkage which does not affect the adhesion of the non-heat-shrinkable sheet edge portion 2, as illustrated in FIG. 3 and FIG. 4, the bottom peripheral surface of the heat-shrinkable sheet portion 1 corresponding to the inner edges of the non-heat-shrinkable sheet edge portion 2 is provided with shrinkage holes 11. The shrinkage holes 11 allow the heat-shrinkable sheet portion 1 to achieve more regular shrinkage and hence will not pull off the adhesion of the non-heat-shrinkable sheet edge portion 2 nor cause deformation of the non-heat-shrinkable sheet edge portion 2. The inner edges of the non-heat-shrinkable sheet edge portion 2 fixed with the heat-shrinkable sheet portion 1 may also be provided with shrinkage notches 22. The shrinkage notches 22 allow more space for the shrinkage and wrinkling of the heat-shrinkable sheet portion 1, hence will not pull off the adhesion of the non-heat-shrinkable sheet edge portion 2 and will not affect the firmness of the adhesion.

To prevent the heat-shrinkable sheet portion 1 from directly contacting the wound, as illustrated in FIG. 1 to FIG. 4, the bottom surface of the heat-shrinkable sheet portion 1 is provided with an isled compound padding 3 which is made of non-woven fabric and polyethylene (PE) barrier film. The non-woven fabric has been immersed in drug solution to kill bacteria and curb inflammation.

As illustrated in FIG. 1 and FIG. 3, the non-heat-shrinkable sheet edge portion 2 is arranged along the bottom peripheral surface of the heat-shrinkable sheet portion 1 and the two portions are fixed as an integral structure, thereby being implemented in the form of an adhesive bandage. As illustrated in FIG. 2 and FIG. 4, the bottom surfaces of the heat-shrinkable sheet portion 1 and the non-heat-shrinkable sheet edge portion 2 are provided with a pair of detachable sheets 4 to protect the isled compound padding 3 and the adhesive layer 21 from damage before use.

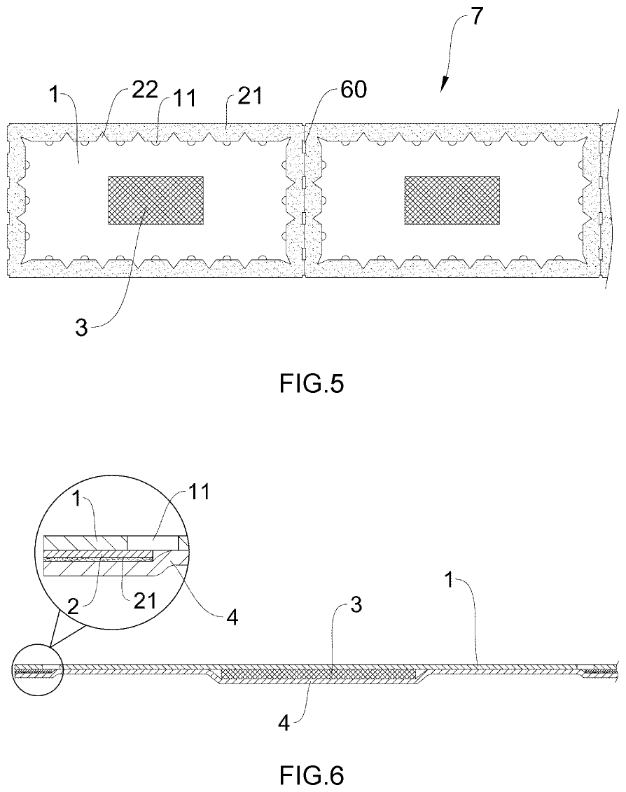

The present invention may also be processed into a roll of multiple pieces of medical dressing as illustrated in FIG. 5 to FIG. 7. In this embodiment, in each piece of medical dressing, the non-heat-shrinkable sheet edge portion 2 is arranged along the bottom peripheral surface of the heat-shrinkable sheet portion 1 and the two portions are fixed as an integral structure. The multiple pieces of the medical dressing are connected together and form a roll and wind around a winding reel 6. A column of tear holes 60 are provided between every two connected pieces of medical dressing so that users can tear off the medical dressing one by one to use.

### The Second Embodiment

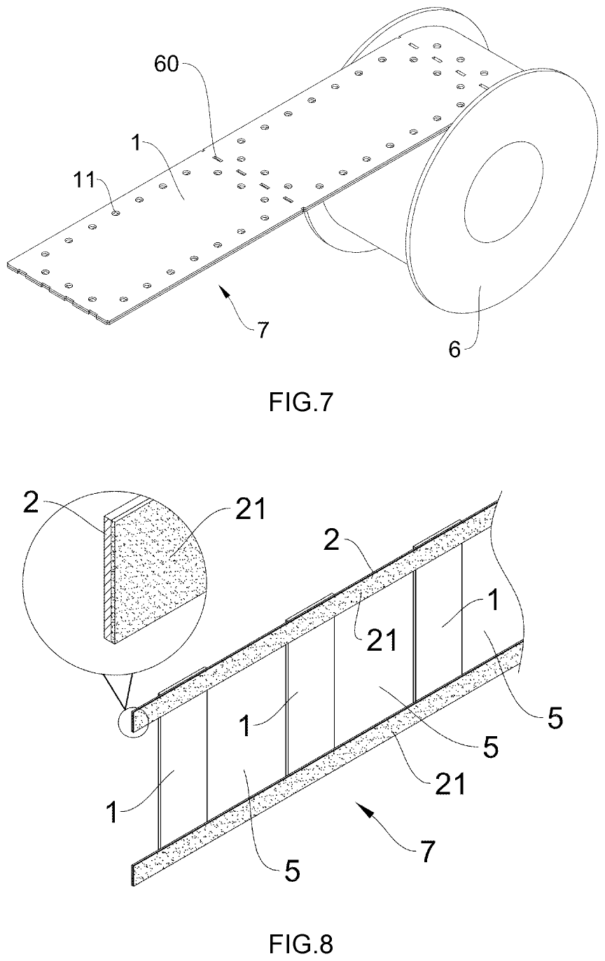

This embodiment and the first embodiment differ in structure. As illustrated in FIG. 8, the second embodiment comprises a plurality of heat-shrinkable sheet portions 1 and a pair of non-heat-shrinkable sheet edge portions 2. The plurality of heat-shrinkable sheet portions 1 are arranged laterally between the pair of non-heat-shrinkable sheet edge portions 2, and are equidistantly spaced apart from one another. The two end portions of each of the heat-shrinkable sheet portions 1 are fixed respectively with the pair of non-heat-shrinkable sheet edge portions 2; there is a separating hole 5 between every two adjacent heat-shrinkable sheet portions 1; the bottom surface of each of the non-heat-shrinkable sheet edge portions 2 is provided with an adhesive layer 21. Therefore, a roll of medical dressing 7 is formed. When the heat-shrinkable sheet portions 1 shrink under heat, a unidirectional shrinkage, i.e. a longitudinal shrinkage, will take place in the second embodiment. The shrinkage in embodiment 1 is centripetal from the four sides. Likewise, the second embodiment may be rolled by providing a winding reel 6 such that the roll of medical dressing 7 is wound around the winding reel 6, as illustrated in FIG. 7.

### The Third Embodiment

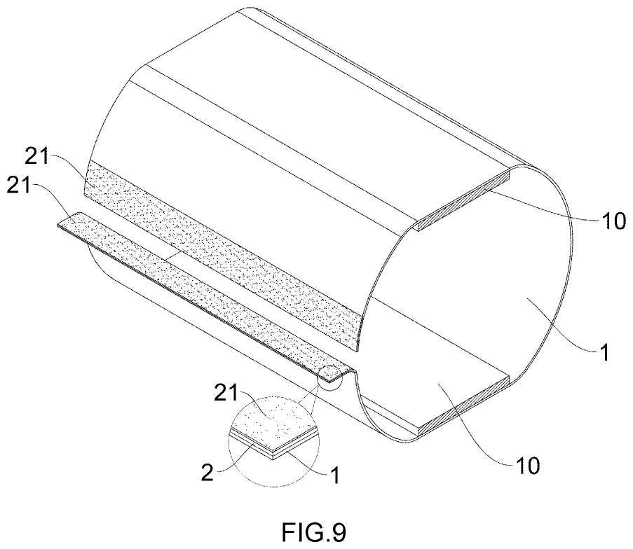

This embodiment differs from the first and second embodiments in the way that the third embodiment wraps and shrinks. As illustrated in FIG. 9, the third embodiment comprises a heat-shrinkable sheet portion 1 and a pair of non-heat-shrinkable sheet edge portions 2. The heat-shrinkable sheet portion 1 is in rectangular shape; the pair of non-heat-shrinkable sheet edge portions 2 are arranged on the two end portions of the heat-shrinkable sheet portion 1 respectively and fixed with the heat-shrinkable sheet portion 1 as a one whole piece. One side of each of the non-heat-shrinkable sheet edge portions 2 is provided with an adhesive layer 21. When the heat-shrinkable sheet portion 1 shrinks under heat, its wrapping structure contracts radially. This embodiment is suitable for treatment of bone fracture. As illustrated in FIG. 9, a pair of clamping plates 10 may be added to clamp and wrap the injured limp.

The above content presents the preferred embodiments of the present invention but does not limit the protection scope thereof. Changes and improvements made by a person skilled in this field of art in accordance with the scope of teachings of the present invention without deviating from the inventive concept of the present invention should also fall within the scope of protection of the present invention.

What is claimed is:

1. A heat-shrinkable medical dressing structure for suture-free and scar-free wound closure, characterized in comprising: a heat-shrinkable sheet portion (1) and a non-heat-shrinkable sheet edge portion (2); the non-heat-shrinkable sheet edge portion (2) is arranged along a bottom peripheral surface of the heat-shrinkable sheet portion (1) and the non-heat-shrinkable sheet edge portion and the heat-shrinkable sheet portion are fixed as an integral structure; a bottom surface of the non-heat-shrinkable sheet edge portion (2) is provided with an adhesive layer (21).

2. The heat-shrinkable medical dressing structure according to claim 1, wherein the bottom peripheral surface of the heat-shrinkable sheet portion (1) corresponding to inner edges of the non-heat-shrinkable sheet edge portion (2) is provided with shrinkage holes (11).

3. The heat-shrinkable medical dressing structure according to claim 2, wherein the inner edges of the non-heat-shrinkable sheet edge portion (2) fixed with the heat-shrinkable sheet portion (1) are provided with shrinkage notches (22).

4. The heat-shrinkable medical dressing structure according to claim 3, wherein a bottom surface of the heat-shrinkable sheet portion (1) is provided with an isled compound padding (3).

5. The heat-shrinkable medical dressing structure according to claim 4, wherein the bottom surfaces of the heat-shrinkable sheet portion (1) and non-heat-shrinkable sheet edge portion (2) are provided with a pair of detachable sheets (4).

6. A heat-shrinkable medical dressing structure for suture-free and scar-free wound closure, characterized in comprising: a plurality of heat-shrinkable sheet portions (1) and a pair of non-heat-shrinkable sheet edge portions (2); the plurality of heat-shrinkable sheet portions (1) are arranged laterally between the pair of non-heat-shrinkable sheet edge portions (2), and are equidistantly spaced apart from one another; two end portions of each of the heat-shrinkable sheet portions (1) are fixed respectively with the pair of non-heat-shrinkable sheet edge portions (2); a separating hole (5) is defined between every two adjacent heat-shrinkable sheet portions (1); a bottom surface of each of the non-heat-shrinkable sheet edge portions (2) is provided with an adhesive layer (21); the heat-shrinkable medical dressing structure is formed as a roll of medical dressing (7).

7. The heat-shrinkable medical dressing structure according to claim 6, wherein a winding reel (6) is provided such that the roll of medical dressing (7) is wound around the winding reel (6).

8. A heat-shrinkable medical dressing structure for suture-free and scar-free wound closure, characterized in comprising: a heat-shrinkable sheet portion (1) and a pair of non-heat-shrinkable sheet edge portions (2); the heat-shrinkable sheet portion (1) is in rectangular shape; the pair of non-heat-shrinkable sheet edge portions (2) are arranged respectively on two end portions of the heat-shrinkable sheet portion (1) and fixed with the heat-shrinkable sheet portion (1) as an integral structure; one side of each of the non-heat-shrinkable sheet edge portions (2) is provided with an adhesive layer (21).
